(19) United States
(12) Patent Application Publication
(10) Pub. No.: US 2012/0270954 A1
(43) Pub. Date: Oct. 25, 2012

(54) METHODS FOR MAINTAINING EYE HEALTH AND AMELIORATING OPTHALMIC MALADIES IN CANINES

(76) Inventors: Yuanlong Pan, Chesterfield, MO (US); Wei Wang, Creve Coeur, MO (US)

(21) Appl. No.: 13/503,953

(22) PCT Filed: Oct. 22, 2010
(86) PCT No.: PCT/US10/02815

§ 371 Date: Jun. 21, 2012

Related U.S. Application Data
(60) Provisional application No. 61/280,175, filed on Oct. 30, 2009.

(51) Int. Cl. A61K 31/015; A61P 29/00; A61P 35/00; A61P 27/02; A61P 27/12

(52) U.S. Cl. 514/763

(57) ABSTRACT

The invention provides novel methods for maintaining eye health and ameliorating ophthalmic maladies in canines by administering to the canines an ophthalmic malady ameliorating amount of one or more carotenes. Generally, the carotenes are administered to the canines in amounts of from about 0.1 to about 5000 mg or in amounts of from about 0.1 to about 500 mg per day to ameliorate maladies such as impaired vision, cataracts, cloudy eye, retinal degeneration, eye injury caused by radiation, reducing or preventing a decline of social interaction, promoting overall health and wellness, improving the quality of life, and extending the prime for canines.

### CROSS REFERENCE TO RELATED APPLICATIONS

This application is a national stage application under 35 U.S.C. §371 of PCT/US2010/002815 filed Oct. 22, 2010, which claims priority to U.S. Provisional Application Ser. No. 61/280,175 filed Oct. 30, 2009, the disclosures of which are incorporated herein by this reference.

### BACKGROUND OF THE INVENTION

1. Field of the Invention

The invention relates generally to methods for maintaining eye health and ameliorating ophthalmic maladies and particularly to methods for using carotenes to maintain eye health and ameliorate ophthalmic maladies in canines.

2. Description of Related Art

Ophthalmic maladies such as cataracts, retinal degeneration, and cloudy eye are common in canines, particularly canines that are outdoors and exposed to excessive sunlight and to aging canines. Methods for counteracting some such maladies are known in the art. WO2009051223 discloses using vascular-adhesion-protein-1 (VAP-1) inhibitors for treating cataracts. US20090082415 discloses using a mixture of topical carriers having carbomer, glycerin, ethylene diamine tetraacetic acid, benzalkonium chloride, balance water, and aldose reductase inhibitors for treating optical complications of diabetes in dogs. U.S. Pat. No. 7,442,711 discloses using N-hydroxy piperidine compounds for the amelioration of cataracts and other ophthalmic diseases. US20060084685 discloses using roflumilast for the treatment of various eye diseases. These methods are, however, insufficient to solve all the problems associated with these and other ophthalmic maladies. There is, therefore, a need for new methods for ameliorating ophthalmic maladies in canines.

### SUMMARY OF THE INVENTION

It is, therefore, an object of the invention to provide methods for ameliorating ophthalmic maladies in canines.

It is another object of the invention to provide methods for maintaining eye health in canines.

It is another object of the invention to provide methods for maintaining or improving vision in canines.

It is a further object of the invention to provide methods for preventing or ameliorating cataracts in canines.

It is another object of the invention to provide methods for preventing or ameliorating cloudy eye in canines.

It is another object of the invention to provide methods for preventing or ameliorating retinal degeneration in canines.

It is another object of the invention to provide methods for preventing or ameliorating canine eye injury in canines caused by radiation.

One or more of these other objects are achieved using novel methods for ameliorating ophthalmic maladies in canines. The methods comprise administering to the canines an ophthalmic malady ameliorating amount of one or more carotenes. The carotenes are administered to the canines in amounts of from about 0.1 to about 5000 milligrams (mg) as desired or in amounts of from about 0.1 to about 500 mg per day. The carotenes accumulate in the canine's eye tissue and ameliorate ophthalmic maladies caused by radiation or free radicals.

Other and further objects, features, and advantages of the present invention will be readily apparent to those skilled in the art.

### DETAILED DESCRIPTION OF THE INVENTION

### Definitions

The term “radiation” means electromagnetic waves having wavelengths that are partially or completely absorbed by carotenes. Generally, carotenes such as β-carotene absorb electromagnetic waves having wavelengths from about 350 to about 550 nanometers (nm), with the best absorption occurring at from about 400 to about 500 nm.

The term “ophthalmic agent” means any compound, composition, or drug, other than carotenes, useful for ameliorating ophthalmic maladies in canines.

The term “in conjunction” means that one or more carotenes and one or more ophthalmic agents are administered to canines (1) together, e.g., in a food composition, or (2) separately at the same or different frequency using the same or different administration routes at about the same time or periodically. “Periodically” means that the agent is administered on a dosage schedule acceptable for a specific agent and that the food is fed to canines routinely as appropriate for the particular animal. “About the same time” generally means that the food and agent are administered at the same time or within about 72 hours of each other. “In conjunction” specifically includes administration schemes wherein an ophthalmic agent is administered for a prescribed period and carotenes are administered indefinitely.

The term “single package” means that the components of a kit are physically associated, in or with one or more containers, and considered a unit for manufacture, distribution, sale, or use. Containers include, but are not limited to, bags, boxes or cartons, bottles, packages of any type or design or material, over-wrap, shrink-wrap, affixed components (e.g., stapled, adhered, or the like), or combinations of any of the foregoing. For example, a single package kit may provide containers of individual compounds and/or compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain, for example, instructions on how to use the kit, or safety or technical information about one or more components of a kit. Examples of information that can be provided as part of a virtual kit include instructions for use; safety information such as material safety data sheets; poison control information; information on potential adverse reactions; clinical study results; dietary information such as food composition or caloric composition; general information on ophthalmic maladies; diseases that affect ophthalmic function, or general information on treatment or preservation of ophthalmic maladies; self-help relating to ophthalmic maladies; caregiver information for those caring for canines with ophthalmic maladies; and use, benefits, and potential side-effects or counter-indications for ophthalmic agents.

The term “health and wellness of an animal” means the complete physical, mental, and social well being of the animal, not merely the absence of disease or infirmity.

The term “quality of life” means the ability to enjoy normal life activities.

The term “extending the prime” means extending the number of years an animal lives a healthy life and not just extending the number of years an animal lives, e.g., an animal would be healthy in the prime of its life for a relatively longer time.

As used throughout, ranges are used herein in shorthand, so as to avoid having to set out at length and describe each and every value within the range. Any appropriate value within the range can be selected, where appropriate, as the upper value, lower value, or the terminus of the range.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “a canine”, “a method”, or “an agent” includes a plurality of such “canines”, “methods”, or “agents”. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Full citations for publications not cited fully within the specification are set forth at the end of the specification.

### The Invention

In one aspect, the invention provides methods for ameliorating ophthalmic maladies in canines. The methods comprise administering to the canines an ophthalmic malady ameliorating amount of one or more carotenes. The invention is based upon the unexpected discovery that carotenes accumulate in canine eye tissue and the knowledge that carotenes are effective for absorbing radiation that damages the eye and for ameliorating damage caused to the eye by free radicals. The discovery contradicts current beliefs that carotenes are not accumulated in canine eye tissue and the knowledge that carotenes do not accumulate in human eye tissue. As disclosed by Yeum (Yeum K J, Taylor A, Tang G, Russell R M. Measurement of carotenoids, retinoids, and tocopherols in human lenses. Invest Ophthalmol. Vis. Sci 1995; 36:2756-61), eye tissue is known to contain xanthophylls such as lutein and zeaxanthin but is known not to contain carotenes such as β-carotene and lycopene. In preferred embodiments, the methods comprise administering to the canines an ophthalmic malady ameliorating amount of one or more carotenes, wherein the carotenes have accumulated in canine eye tissue.

Since eye tissue does not synthesize carotenes, any carotenes in the eye must be derived from external sources, e.g., the diet. For known eye tissue, i.e., human eye tissue, the consumption of carotenes produces a short term systemic elevation of carotenes. In contrast, canine eye tissue selectively absorbs carotenes and keeps carotenes within the eye tissue, i.e., canine eye tissue accumulates carotenes. These carotenes remain in the eye even when carotenes are not being consumed regularly and the systemic level of carotenes has declined. This accumulation of carotenes provides protection against radiation and free radicals and ameliorates ophthalmic maladies in the canines caused by radiation and free radicals. Therefore, at least for canines, the surprising discovery that carotenes accumulate in canine eye tissue gives the canine an advantage over other animals because the accumulated carotenes absorb harmful electromagnetic radiation and scavenges free radicals that are known to cause ophthalmic maladies.

The carotenes accumulate in the eyes, including eye tissue susceptible to damage from radiation and free radicals. The carotenes accumulate in the cortical eye tissue, the nuclear eye tissue, or both. Carotenes that accumulate in such tissue provide protection to the lens and related tissue by preventing damage by radiation and free radicals.

The ophthalmic maladies ameliorated by the invention are any maladies caused by radiation or free radical damage to the eye. In one embodiment, the maladies are maladies caused by radiation. Maladies known to be caused by radiation include cataracts, radiation retinopathy, photokeratitis, photoconjunctivitis, pterygium, pinguecula, and cancer. In another embodiment, the maladies are maladies caused by free radicals. Maladies known to be caused by free radicals include cloudy eye, cataracts, and retinal degeneration.

The carotenes useful in the invention are any carotenes suitable for administration to canines. Such carotenes and their sources are known to skilled artisans. Generally, carotenes are obtained from any suitable source, synthetic, natural, and combinations thereof. A preferred source of carotenes is dietary carotenes contained in many fruits and vegetables, e.g., carrots, pumpkins, sweet potatoes, spinach, Vietnamese gac, palm oil, mangoes, broccoli, palayas yams, kale, beet greens, winter squash, chard, and the like. Other sources include synthetic carotenes, e.g., as dietary supplements in tablet, capsule, and similar forms.

In preferred embodiments, the carotenes are α-carotene, β-carotene, γ-carotene, δ-carotene, ε-carotene, lycopene, and combinations thereof. Preferably the carotenes are β-carotene, lycopene, and combinations thereof. Most preferably, the carotenes are β-carotene.

The carotenes are administered to the canines in any amount that is effective for ameliorating ophthalmic maladies. In various embodiments, the carotenes are administered to the canine in amounts of from about 0.1 to about 5000 mg as desired or needed, preferably from about 0.5 to about 3000 mg, most preferably from about 1 to about 1000 mg. In other embodiments, the carotenes are administered in amounts of from about 0.1 to about 500 mg per day, preferably from about 0.5 to about 250 mg per day, most preferably from about 1 to about 100 mg per day. The amount may vary within the range depending on the malady and the characteristics of the canine.

The carotenes are administered to the canines using any suitable method, e.g., orally. In various embodiments, administration is on an as-needed or as-desired basis of varying or regular frequency depending on the intended use. In other embodiments, administration is on a regular or scheduled basis. Administration on a regular or scheduled basis provides the canines with a regular and consistent dose of carotenes that keeps the level of carotenes in the eye relatively constant. Regular or scheduled administration can be once monthly, once weekly, once daily, or more than once daily. Similarly, administration can be every other day, week, or month, every third day, week, or month, every fourth day, week, or month, and the like. Administration can be multiple times per day. Such administration can depend on the age, size, health, malady, and the like for the canine. In some embodiments, carotenes or comestible ingredients containing carotenes are applied to or admixed with typical foods and drinks and administered to the canines, e.g., carotenes are an integral part of a canine pet food. In one embodiment, administration is part of a dietary regimen. For example, such a dietary regimen comprises the regular ingestion of one or more carotenes or any comestible ingredient containing such carotenes in an amount effective for ameliorating ophthalmic maladies. In preferred embodiments, the carotenes are administered to the canines as part of a food composition or in the form of a dietary supplement. Preferably, the carotenes are administered to the canines as part of a wet or dry pet food composition, e.g., kibbles or wet canned pet food. In a preferred embodiment, the carotenes are administered on an extended regular basis, preferably a daily basis.

In various embodiments, the canines are wolves, foxes, jackals, coyotes, and dogs, preferably dogs, most preferably domestic dogs.

Carotene administration, including administration as part of a dietary regimen, can span a period ranging from parturition through the life of the canine. In certain embodiments, the canine is a young, growing, or adult canine. In preferred embodiments, the canine is an aging canine susceptible to or suffering from ophthalmic maladies characteristic of aging. In other embodiments, the canine is a canine that is likely to be exposed to excess radiation that will adversely impact the eye, e.g., a hunting dog or a working dog.

In one embodiment, the methods further comprise administering the carotenes in conjunction with one or more ophthalmic agents in an amount effective for ameliorating ophthalmic maladies as defined herein. In a particular embodiment, the carotenes are administered in a pharmaceutical composition that includes one or more ophthalmic agents along with one or more carotenes. In a preferred embodiment, the carotenes are administered to the animal on a daily basis, preferably as part of a food composition or in a dietary supplement, and the ophthalmic agents are administered on an as prescribed basis, generally in the form of a pharmaceutical composition such as a pill. Such agents include avastin (e.g., for radiation retinopathy); antibiotics; anti-inflammatory compounds (e.g., COX-2 inhibitors); tranquilizers; antioxidants such as resveratrol, proanthocyanidins, anthocyanins, carotenoids (e.g., lutein, zeaxanthin, astaxanthin, cryptoxanthin, and lycopene); bioflavonoids (e.g., taxifolin, naringenin, and hesperetin); glutathione; catechin; epicatechin; epigallocatechin; epigallocatechin gallate; epicatechin gallate; and lipoic acid; and vitamins, e.g., vitamin A, vitamin E, and vitamin C. Skilled artisans can determine the proper type and amount of ophthalmic agents to administer to the canines based upon their age, size, health, malady, and the like for the canine.

Methods that ameliorate ophthalmic maladies have many beneficial effects for canines. Many day-to-day life functions are dependent upon or related to ophthalmic function. Eliminating or lessening the impact and severity of ophthalmic maladies that interfere with normal ophthalmic function, particularly those that impair vision, improve many aspects of life. For example, ameliorating ophthalmic maladies is related to (1) maintaining eye health; (2) maintaining or improving vision; (3) preventing or ameliorating cataracts; (4) preventing or ameliorating cloudy eye; (5) preventing or ameliorating canine eye injury caused by radiation; (6) preventing or ameliorating retinal degeneration; (7) reducing or preventing a decline of social interaction; (8) promoting overall health and wellness; (9) improving the quality of life; and (10) extending the prime. Therefore, in various aspects, the invention provides methods for affecting various ophthalmic related functions adversely affected by these ophthalmic maladies. In all these aspects, the benefit results from an accumulation of carotenes in the eyes of the canine, i.e., in the eye tissue exposed to radiation and susceptible to free radical damage.

In one such aspect, the invention provides methods for maintaining eye health in canines comprising administering to the canines an eye health maintaining amount of one or more carotenes. The carotenes maintain overall eye health by preventing ophthalmic maladies in canines, particularly those caused by radiation and by free radicals.

In another such aspect, the invention provides methods for maintaining or improving vision in canines comprising administering to the canines a vision maintaining or improving amount of one or more carotenes. The carotenes absorb radiation and scavenge free radicals that are at least partially responsible for causing ophthalmic maladies that impair vision. With no or less severe ophthalmic maladies, the canines have better vision.

In another aspect, the invention provides methods for preventing or ameliorating cataracts in canines comprising administering to the canines a cataract preventing or ameliorating amount of one or more carotenes. The carotenes absorb radiation and scavenge free radicals that are at least partially responsible for causing cataracts in canines. With no or less severe cataracts, the canines have better vision and overall eye health.

In another aspect, the invention provides methods for preventing or ameliorating cloudy eye in canines comprising administering to the canines a cloudy eye preventing or ameliorating amount of one or more carotenes. The carotenes absorb radiation and scavenge free radicals that are at least partially responsible for causing cloudy eye in canines. With no or less severe cloudy eye, the canines have better vision and have a more healthy and appealing appearance.

In a further aspect, the invention provides methods for preventing or ameliorating canine eye injury caused by radiation comprising administering to the canines an eye injury preventing or ameliorating amount of one or more carotenes. The carotenes absorb radiation that damages eye tissue and causes injury to the eye.

In another aspect, the invention provides methods for preventing or ameliorating retinal degeneration in canines comprising administering to the canines a retinal degeneration preventing or ameliorating amount of one or more carotenes. The carotenes absorb radiation and scavenge free radicals that are at least partially responsible for causing retinal degeneration in canines. With no or less severe retinal degeneration, the canines have better vision and have a more healthy and appealing appearance.

In yet another aspect, the invention provides methods for reducing or preventing a decline of social interaction in canines comprising administering to the canines a social interaction decline reducing or preventing amount of one or more carotenes. The carotenes absorb radiation and scavenge free radicals that are at least partially responsible for impairing vision in canines. With better vision, the canines are more socially interactive, particularly when participating in activities that require good vision, e.g., fetch. The methods ensure that the canines remain involved in playtime, participate in group activities, interact with caregivers, and the like.

In one aspect, the invention provides methods for promoting the health and wellness of canines comprising administering to the canines a health and wellness promoting amount of one or more carotenes. The carotenes improve eye health and vision and improve the canine's overall health and wellness.

In another aspect, the invention provides methods for improving the quality of life for canines comprising administering to the canines a quality of life improving amount of one or more carotenes. The carotenes improve eye health and vision and contribute to the quality of life of the canines.

In a further aspect, the invention provides methods for extending the prime for canines comprising administering to the canines a prime extending amount of one or more carotenes. The methods extend the time the canines have good vision and delay the onset of ophthalmic maladies so that the canines have a longer period during their life wherein they have good vision and related ophthalmic functions.

In the methods for maintaining eye health, maintaining or improving vision, preventing or ameliorating cataracts, preventing or ameliorating cloudy eye, preventing or ameliorating retinal degeneration, preventing or ameliorating canine eye injury caused by radiation, reducing or preventing a decline of social interaction, promoting overall health and wellness, improving the quality of life, and extending the prime, the amount of carotenes used in these methods are the same as the amounts or within the ranges given herein for ameliorating ophthalmic maladies. Administration routes, specific compounds, and other parameters are the same as described herein for ameliorating ophthalmic maladies.

In a further aspect, the invention provides kits suitable for administering carotenes to canines to ameliorate ophthalmic maladies. The kits comprise in separate containers in a single package or in separate containers in a virtual package, as appropriate for the kit component, instructions for how to use carotenes to ameliorate ophthalmic maladies and at least one of (1) one or more carotenes; (2) one or more comestible ingredients containing carotenes; (3) one or more comestible ingredients compatible with carotenes; (4) one or more ophthalmic agents; (5) instructions for how to combine or prepare the carotenes and any other ingredients provided in the kit for administration to canines; and (6) a device for administering the combined or prepared kit components to canines. The components are each provided in separate containers in a single package or in mixtures of various components in different packages. In preferred embodiments, the kits comprise the instructions and one or more carotenes or comestible ingredients containing carotenes. The kits may comprise the ingredients in various combinations.

In another aspect, the invention provides a means for communicating information about or instructions for one or more of (1) using carotenes for ameliorating ophthalmic maladies; (2) using carotenes in conjunction with ophthalmic agents; (3) admixing carotenes with other materials to produce a composition suitable for ameliorating ophthalmic maladies; (4) using the kits of the invention for ameliorating ophthalmic maladies; (5) benefits of using carotenes for ameliorating ophthalmic maladies; and (6) administering carotenes to canines. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the present invention provides packages comprising one or more carotenes and a label affixed to the packages containing a word or words, picture, design, acronym, slogan, phrase, or other device, or combination thereof, that indicates that the contents of the package contains carotenes that are suitable for one or more of ameliorating ophthalmic maladies; maintaining eye health; maintaining or improving vision; preventing or ameliorating cataracts; preventing or ameliorating cloudy eye; preventing or ameliorating retinal degeneration; preventing or ameliorating canine eye injury caused by radiation; reducing or preventing a decline of social interaction; promoting overall health and wellness; improving the quality of life; and extending the prime for canines. Typically, such device comprises the words ‘prevents damage caused by ultraviolet light, “maintains eye health, “prevents eye damage caused by radiation”, “helps prevent cataracts”, “helps prevent cloudy eye in dogs”, or an equivalent expression printed on the package. Any package or packaging material suitable for containing the carotenes is useful in the invention, e.g., a bag, box, bottle, can, pouch, and the like manufactured from paper, plastic, foil, metal, and the like. In a preferred embodiment, the package contains a food composition adapted for a canine that supplies the required amount of carotenes to the canine, e.g., a pet food composition formulated for canines.

In another aspect, the invention provides for use of one or more carotenes to prepare a medicament for one or more of ameliorating ophthalmic maladies; maintaining eye health; maintaining or improving vision; preventing or ameliorating cataracts; preventing or ameliorating cloudy eye; preventing or ameliorating retinal degeneration; preventing or ameliorating canine eye injury caused by radiation; reducing or preventing a decline of social interaction; promoting overall health and wellness; improving the quality of life; and extending the prime for canines. Generally, medicaments are prepared by admixing carotenes with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to canines.

### EXAMPLES

The invention can be further illustrated by the following examples, although it will be understood that the examples are included merely for purposes of illustration and are not intended to limit the scope of the invention unless otherwise specifically indicated.

### Example 1

Eye samples were collected from seven (7) dogs. Eye balls were removed within 1 hour of death at necropsy, stored in individual containers at 4° C., and stored on wet ice for dissection within one day. The eyes were dissected to take out lenses. The lenses were separated into two layers: (1) an outer, soft cortical/epithelial layer and (2) an inner, hard nuclear layer. The layers were analyzed for carotenoids, vitamin C, and vitamin E following standard procedures given in Craft N E, Haitema T B, Garnett K M, Fitch K A, Dorey C K. Carotenoid, tocopherol, and retinol concentrations in elderly human brain. J Nutr. Health Aging 2004; 8:156-62. The results are given in Table 1.

TABLE 1
Antioxidant Levels in Different Layers of Dog Lens

| Variables | Cortical/Epithelial Layer (C) | Nuclear Layer (N) |
|---|---|---|
| β-Carotene (ng/g) | 1.74 | 2.74 |
| Vitamin C (μg/g) | 112.38 | 23.59 |
| Vitamin E (μg/g) | 0.93 | 0.64 |

Nanograms (ng);
grams (g).

Referring to Table 1, the data show that all nuclear layer samples accumulated β-carotene and that six out of seven cortical layer samples accumulated β-carotene.

TABLE 2
Concentrations of β-Carotene in
Different Layers of Individual Dog Lens

| | | | | | Lens | |
|---|---|---|---|---|---|---|
| | | | | Lens | Weight | β-Carotene |
| Dog # | Age | Breed | Sex | Layers | (grams) | (ng/g) |
| 1 | 15.50 | BEA | M/n | C | 0.23 | nd* |
| | | | | N | 0.38 | 0.79 |
| 2 | 13.55 | BEA | M/n | C | 0.22 | 1.06 |
| | | | | N | 0.32 | 0.60 |
| 3 | 13.41 | LAB | F/s | C | 0.57 | 0.73 |
| | | | | N | 0.27 | 1.26 |
| 4 | 14.22 | LAB | M/n | C | 0.45 | 0.71 |
| | | | | N | 0.23 | 0.79 |
| 5 | 12.79 | Man T | F/s | C | 0.37 | 1.35 |
| | | | | N | 0.19 | 1.60 |
| 6 | 6.10 | SIB HUSKY | M/n | C | 0.25 | 4.26 |
| | | | | N | 0.26 | 8.17 |
| 7 | 9.31 | LAB | F/s | C | 0.37 | 2.35 |
| | | | | N | 0.26 | 5.99 |

*Detection limit for carotenoids is 0.2 ng/g.
*Neutered (n); Spayed (s).

Referring to Table 2, the data show that different types and sexes of dogs accumulate β-carotene in different types of eye tissue. In contrast, previous studies (See, Bates C J, Chen S J, Macdonald A, Holden R. Quantization of vitamin E and a carotenoid pigment in cataractous human lenses, and the effect of a dietary supplement. Int. J Vitam. Nutr. Res 1996:66:316-21; Bernstein P S, Khachik F, Carvalho L S, Muir G J, Zhao D Y, Katz N B. Identification and quantization of carotenoids and their metabolites in the tissues of the human eye. Exp. Eye Res 2001:72:215-23; Yeum K J, Taylor A, Tang G, Russell R M. Measurement of carotenoids, retinoids, and tocopherols in human lenses. Invest Ophthalmol. Vis. Sci 1995:36:2756-61; and Yeum K J, Shang F M, Schalch W M, Russell R M, Taylor A. Fat-soluble nutrient concentrations in different layers of human cataractous lens. Curr. Eye Res 1999; 19:502-5) showed that (3-carotene was not detected or accumulated in human lenses even though humans regularly consume β-carotene in their diets. These data in Table 2 show that canine eye tissue is very unique because it accumulates β-carotene. β-Carotene as a yellow-pigment blocks electromagnetic radiation that damages the eyes. In addition, β-carotene is a powerful antioxidant that prevents and reduces free radicals formation in the eyes. Therefore, β-carotene plays a unique and critical role in maintaining eye health and ameliorating ophthalmic maladies in canines such as dogs.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

1. A method for ameliorating ophthalmic maladies in canines comprising administering to the canines an ophthalmic malady ameliorating amount of one or more carotenes.

2. The method of claim 1 wherein the carotenes have accumulated in eye tissue.

3. The method of claim 2 wherein the carotenes have accumulated in the cortical eye tissue, the nuclear eye tissue, or both.

4. The method of claim 1 wherein the maladies are caused by radiation.

5. The method of claim 4 wherein the maladies are one or more of cataracts, radiation retinopathy, photokeratitis, photoconjunctivitis, pterygium, pinguecula, and cancer.

6. The method of claim 1 wherein the maladies are caused by free radicals.

7. The method of claim 6 wherein the maladies are one or more of cloudy eye, cataracts, and retinal degeneration.

8. The method of claim 1 wherein the carotenes are α-carotene, β-carotene, γ-carotene, δ-carotene, ε-carotene, lycopene, and combinations thereof.

9. The method of claim 1 wherein the carotenes are β-carotene, lycopene, and combinations thereof.

10. The method of claim 1 wherein the carotene is β-carotene.

11. The method of claim 1 wherein the carotenes are administered to the canines in amounts of from about 0.1 to about 5000 mg.

12. The method of claim 1 wherein the carotenes are administered to the canines in amounts of from about 0.1 to about 500 mg per day.

13. The method of claim 1 wherein the canines are dogs.

14. The method of claim 1 wherein the carotenes are administered to the canines on an extended regular basis.

15. The method of claim 1 wherein the carotenes are administered to the canines on a daily basis.

16. The method of claim 1 wherein the canines are aging canines.

17. The method of claim 1 further comprising administering the carotenes in conjunction with one or more ophthalmic agents in an amount effective for ameliorating ophthalmic maladies.

18. (canceled)

19. (canceled)

20. (canceled)

21. A method for preventing or ameliorating cloudy eye in canines comprising administering to the canines a cloudy eye preventing or ameliorating amount of one or more carotenes.

22. A method for preventing or ameliorating retinal degeneration in canines comprising administering to the canines a retinal degeneration preventing or ameliorating amount of one or more carotenes.

23. (canceled)

24. (canceled)

25. (canceled)

26. (canceled)

27. (canceled)

28. (canceled)

29. (canceled)

30. (canceled)

31. (canceled)

32. (canceled)
